(19) United States
(12) Patent Application Publication
(10) Pub. No.: US 2020/0055054 A1
(43) Pub. Date: Feb. 20, 2020

(54) CLAMPING SYSTEM FOR A MICROFLUIDIC ASSEMBLY

(71) Applicant: President and Fellows of Harvard College, Cambridge, MA (US)

(72) Inventors: Kambez Hajipouran Benam, Cambridge, MA (US); Donald E. Ingber, Boston, MA (US); Richard Novak, Jamaica Plain, MA (US)

(21) Appl. No.: 15/781,052

(22) PCT Filed: Dec. 2, 2016
(86) PCT No.: PCT/US2016/064813

§ 371 Date: Jun. 1, 2018

Related U.S. Application Data
(60) Provisional application No. 62/263,206, filed on Dec. 4, 2015.

(51) Int. Cl. B01L 9/00; C12M 3/06; C12M 3/00; C12M 1/12

(52) U.S. Cl. CPC B01L 9/50; B01L 9/52; B01L 2200/027; C12M 23/46; C12M 25/02; C12M 23/16

(57) ABSTRACT

A clamping system for a microfluidic device includes a compression plate engaging a side of a microfluidic device. A compression device provides compressive forces. The compression device is operatively connected to the compression plate such that the compressive forces create a substantially uniform pressure on the side of the microfluidic device.

### CROSS-REFERENCE TO RELATED APPLICATIONS

This application claims benefit under 35 U.S.C. § 119(e) of U.S. Provisional Application No. 62/263,206, filed Dec. 4, 2015, the contents of which are incorporated herein by reference in their entirety.

### GOVERNMENT SUPPORT

This invention was made with government support under grant no. W911NF-12-2-0036 awarded by U.S. Department of Defense, Advanced Research Projects Agency. The government has certain rights in the invention.

### TECHNICAL FIELD

The present invention relates to cell culture systems and fluidic systems. More specifically, the invention relates to a clamping system for microfluidic device.

### BACKGROUND

In microfluidic devices that are designed for experimentation on cells, there is typically an “active area” at which the cell culturing and experimentation are performed. Other areas in the device serve other functions. It is often desirable to constrain the cells to the active area and avoid cells in the other areas. In one exemplary microfluidic device having a membrane that separates two microchannels, it is desirable to have the cells retained in the membrane region of the device, where cells can communicate through the membrane. On the other hand, it is desirable to avoid cells in the various fluid inlet and outlet channels that lead to and from the membrane region.

Furthermore, it is desirable to limit the biological communication within microdevices to the active region at which the cell layers are separated by a porous membrane. Fluidic seals are used when coupling non-bondable materials, such as membrane, to contain cell in the channels and chambers to minimize cell escape and growth between membranes and the sealing materials. Such cell escape or growth can cause unclear tissue boundaries, variability in bioassays and growth rates of tissue, or cell escape into the surrounding fluidic channels.

Seals can include holes in the membrane dividing the top and bottom halves of microfluidic devices made of polymeric organosilicon compounds to provide areas of bonding to seal in an unbounded membrane. However, such approaches are sensitive to bonding conditions. Screw clamps can also be used to overcome delamination problems in microfluidic devices. However, prior clamping approaches cause uneven pressure to be applied to the microfluidic device.

The present invention solves many of the problems associated with the prior art systems by providing for robust fluidic seals in fluidic devices, when coupling non-bondable materials, so that cells in the channels and chambers are contained and so that cell escape and growth between membranes and sealing materials is minimized. The modularity and interchangeability of fluidic components is also improved to allow for more versatile experimentation when using microfluidic devices, including improved handling of fluidic devices during cell seeding, microfluidic device operation, and experimentation.

### SUMMARY

According to one aspect of the present invention, a clamping system for a microfluidic device includes a compression plate engaging a side of a microfluidic device. A compression device provides compressive forces. The compression device is operatively connected to the compression plate such that the compressive forces create a substantially uniform pressure on the side of the microfluidic device.

According to another aspect of the present invention, a clamping system for a microfluidic device includes a base and a plurality of elongated side support structures. Each side support structure protrudes upwardly from an edge of the base. At least two of the plurality of elongated side support structures each includes a vertical post. A top cover rigidly connects the at least two of the plurality of elongated side structures. The at least two of the plurality of elongated side structures connect at opposing sides of the top cover. A compression plate is movably coupled to at least two of the vertical posts such that the compression plate is vertically slidable between the base and the top cover along a long axis of each of the at least two vertical posts. The compression plate includes at least one inlet access hole that substantially aligns with a corresponding fluid inlet on a microfluidic device and at least one outlet access hole that substantially aligns with a corresponding fluid outlet on the microfluidic device. A compression device provides compressive forces along each of the long axes of the at least two vertical posts. The compression device is operatively connected to the compression plate such that the compressive forces are applied directly to the compression plate. The compression plate is further operative to slide upwardly along the at least two vertical posts until the compression device is in a fully compressed state and operative to slide downwardly along the at least two vertical posts so that the compression plate applies a substantially uniform pressure to a top surface of a microfluidic device positioned between the compression plate and the base.

In a yet another aspect of the present invention, a microfluidic system comprises a microfluidic device including a top surface, a bottom surface, at least one microchannel, a fluid inlet, and a fluid outlet. A clamp system includes (i) a moveable compression plate for engaging the top surface of the microfluidic device, and (ii) a compression device for urging the moveable compression plate downwardly against the top surface of the microfluidic device to place a substantially uniform pressure on the top surface. The compression plate includes an inlet access hole that substantially aligns with the fluid inlet on the microfluidic device and an outlet access hole that substantially aligns with the fluid outlet on the microfluidic device.

In a yet another aspect of the present invention, a microfluidic system comprises a microfluidic device having a top surface, a bottom surface, at least one microchannel, a fluid inlet, and a fluid outlet. A clamp system includes (i) a moveable compression plate for engaging the top surface of the microfluidic device, and (ii) a compression device for urging the moveable compression plate downwardly against the top surface of the microfluidic device to place a substantially uniform pressure on the top surface. The base includes a viewing window that permits imaging of a region of the at least one microchannel.

In a yet another aspect of the present invention, a microfluidic system comprises a microfluidic device including a top part, a bottom part, a membrane between the top and bottom parts, and at least one microchannel at least partially defined by the membrane. A clamp system includes (i) a moveable compression plate for engaging the top part of the microfluidic device in a closed state and being released from the top surface in an opened state, and (ii) a compression device for controllably moving the moveable compression plate between the closed state during operation of the microfluidic device and the opened state allowing the top part to be removed from at least one of the membrane and the bottom part.

In yet another aspect of the present invention, a method of clamping a microfluidic device comprises providing (i) a microfluidic device comprising a side with ports in fluidic communication with at least one internal channel, and (ii) a clamping system for clamping said microfluidic device. The clamping system comprises a compression plate engaging a side of the microfluidic device and a compression device for providing compressive forces. The compression device is operatively connected to the compression plate. Compressive forces are applied with said compression device such that substantially uniform pressure is created on the side of the microfluidic device.

In yet another aspect of the present invention, a method of cell culture comprises positioning a plurality of clamped microfluidic devices side-by-side next to each other on a support. Each clamped microfluidic device comprises (i) a microfluidic device comprising a side with ports in fluidic communication with at least one internal channel, said channel comprising cells to be cultured and (ii) a clamping system comprising a compression plate engaging a side of the microfluidic device and a compression device for providing compressive forces. The compression device is operatively connected to the compression plate. Culture media flows through the channels of each clamped microfluidic device.

In yet another aspect of the present invention, a clamping system for a microfluidic device comprises a compression plate engaging a biocompatible surface on a microfluidic device. A compression device provides compressive forces. The compression device is operatively connected to the compression plate such that the compressive forces uniformly seal an open region of the microfluidic device.

Additional aspects of the invention will be apparent to those of ordinary skill in the art in view of the detailed description of various embodiments, which is made with reference to the drawings, a brief description of which is provided below.

### BRIEF DESCRIPTION OF THE DRAWINGS

FIG. 1 illustrates an exemplary microfluidic device with a membrane region having cells thereon that may be used with the present invention.

FIG. 2 is a cross-section of the microfluidic device taken along line 2-2 of FIG. 1, illustrating the membrane separating the first microchannel and the second microchannel.

FIGS. 3A-3C illustrate an exemplary clamping system including isometric, top, and side views that may be used for microfluidic devices and microfluidic systems according to one embodiment.

FIGS. 4A and 4B illustrate exemplary clamping systems that pivot about a base that may be used for microfluidic devices and microfluidic systems according to certain embodiments.

FIGS. 5A-5C illustrate an exemplary clamping system including isometric, top, and side views that may be used for microfluidic devices and microfluidic systems according to one embodiment.

FIG. 6 illustrates an exemplary microfluidic system including a clamping device and a microfluidic device according to one embodiment.

FIGS. 7A and 7B illustrate an exemplary microfluidic system including a clamping device in a closed state and in an open state according to one embodiment.

FIG. 8 illustrates an exemplary microfluidic system including a clamping device, a microfluidic device, and fluid connectors connected to the microfluidic device according to one embodiment.

FIGS. 9A and 9B illustrate an exemplary microfluidic device including a clamping device, a microfluidic device, and fluid connectors and needles connected to the microfluidic device according to one embodiment.

FIGS. 10A and 10B illustrate an exemplary clamp system farm that may be used for microfluidic devices and microfluidic systems according to one embodiment.

FIG. 11 illustrates an exemplary clamp system farm as part of a cell culture incubator that may be used for microfluidic devices and microfluidic systems according to one embodiment.

While the invention is susceptible to various modifications and alternative forms, specific embodiments have been shown by way of example in the drawings and will be described in detail herein. It should be understood, however, that the invention is not intended to be limited to the particular forms disclosed. Rather, the invention is to cover all modifications, equivalents, and alternatives falling within the spirit and scope of the invention as defined by the appended claims.

### DETAILED DESCRIPTION

While this invention is susceptible of embodiment in many different forms, there is shown in the drawings and will herein be described in detail preferred aspects of the invention with the understanding that the present disclosure is to be considered as an exemplification of the principles of the invention and is not intended to limit the broad aspect of the invention to the aspects illustrated. For purposes of the present detailed description, the singular includes the plural and vice versa (unless specifically disclaimed); the word “or” shall be both conjunctive and disjunctive; the word “all” means “any and all”; the word “any” means “any and all”; and the word “including” means “including without limitation.”

The functionality of cells and tissue types (and even organs) can be implemented in one or more microfluidic devices or “chips” that enable researchers to study these cells and tissue types outside of the body while mimicking much of the stimuli and environment that the tissue is exposed to in-vivo. It can also be desirable to implement these microfluidic devices into interconnected components that can simulate groups of organs or tissue systems. Preferably, the microfluidic devices can be easily inserted and removed from an underlying fluidic system that connects to these devices in order to vary the simulated in-vivo conditions and organ systems.

The present disclosure relates to improvements in fluidic systems that include a fluidic device, such as a microfluidic device, and an improved clamping system. Fabrication of robust fluidic seals is desirable in micro- and meso-fluidic devices when coupling non-bondable materials, including membranes. It is also desirable to improve the modularity and the interchangeability of fluidic components to enable versatile experimentation. Robust fluidic seals are particularly desirable in microfluidic devices or “chips” for studying the functionality of cells and tissue types. Such seals contain the cells in the channels and chambers of the microfluidic device and minimize cell escape and growth between membranes and the sealing material, which can cause unclear tissue boundaries, variability in bioassays and growth rates of tissue, as well as cell escape into surrounding microfluidic channels. By using the described clamping systems to provide the robust seals, delamination of the membrane in a microfluidic device and fluidic path changes are minimized, which reduces negative biological influences and device failure that might otherwise occur due to the unwanted spread and growth of the cells in undesirable areas or channels of the fluidic device.

Furthermore, the present disclosure describes a clamping system that improves the handling of fluidic devices, such as “chips” for cells and tissue types, during cell seeding, device operation, and experimentation. For example, the clamping systems can act as a manifold that improves the assembly, handling, and operation of the fluidic device or devices contained within the clamping system. A user of the described clamping systems can have the option to change parts of the microfluidic device while an experiment is ongoing (e.g. replace the top compartment with a new one if cells grown on the side walls or the roof of a top channel). A user can also, for example, open up the microfluidic device to expose the cells that are in culture to external stimuli (e.g. exposure to aerosolized particles). Furthermore, the described clamping system can also be integrated with a cell culture medium reservoir or a fluidic pump. It is also contemplated that a co-culture of cells on opposite side of a microfluidic device membrane can be carried out without the need for external support options to keep the membrane level. In addition, the improved clamping system allows a user to quickly and easily change the fluidic connections without terminating an experiment/cell culture (e.g. assays involving circulating immune cells can be carried out using appropriate connections).

A clamping system is described for fluidic devices, such as microfluidic devices, that distributes force substantially uniformly across the surface of a material or materials forming the microfluidic or mesofluidic device to sandwich the components that might not otherwise produce a robust fluidic seal through bonding methods. The pressure caused by the force is of a level that does not distort the channels or membranes, and thus, the desirable pressure for providing a robust fluidic seal varies based on several factors, including the membrane material and geometry along with the material and geometry of the microfluidic device.

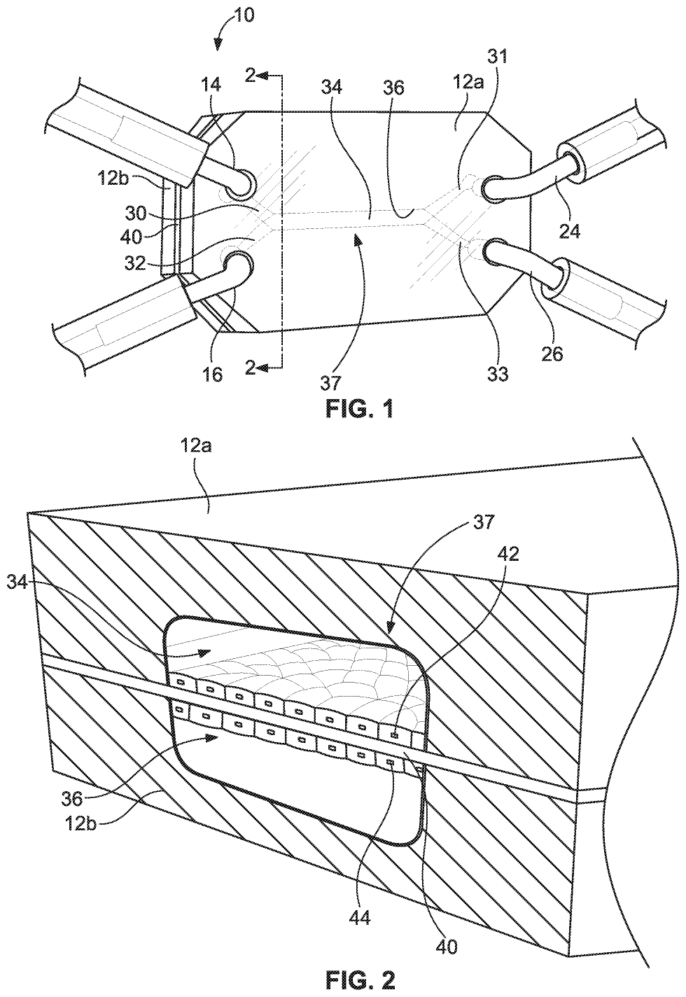

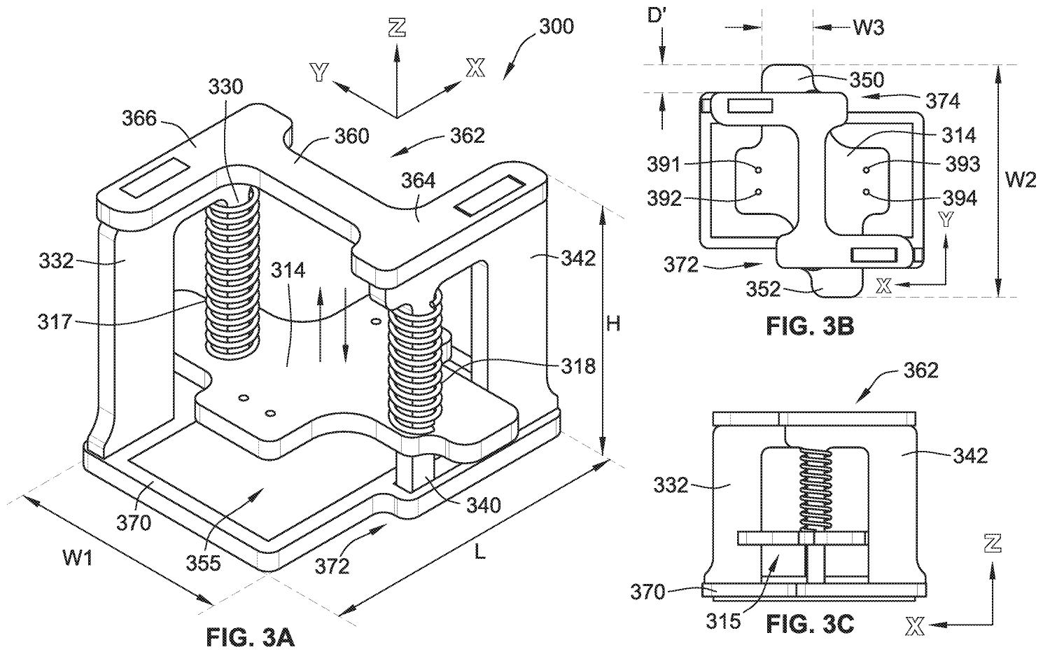

Referring now to FIGS. 1 and 2, one type of an organ-on-chip (“OOC”) device 10 is illustrated to which the described clamp systems can be applied. The OOC device 10 includes a body 12 that is typically comprised of an upper body segment 12a and a lower body segment 12b. The upper body segment 12a and the lower body segment 12b are preferably made of a polymeric material, such as PDMS (poly-dimethylsiloxane), polycarbonate, cyclo-olefin polymers, polyurethanes, styrene derivatives like SEBS (Styrene Ethylene Butylene Styrene) or other polymer materials. In some aspects, the upper and lower body segments are made of extracellular matrix (ECM) scaffolds, like collagen, gelatin, etc. The upper body segment 12a includes a first fluid inlet 14 and a second fluid inlet 24. A first fluid path for a first fluid includes the first fluid inlet 14, a first seeding channel 30, an upper microchannel 34, an exit channel 31, and then the first fluid outlet 24. A second fluid path for a second fluid includes the second fluid inlet 16, a first seeding channel 32, a lower microchannel 36, an outlet channel 33, and then the second fluid outlet 26.

Referring to FIG. 2, a membrane 40 extends between the upper body segment 12a and the lower body segment 12b. The membrane 40 is preferably an inert, polymeric, micro-molded membrane having uniformly distributed pores with sizes normally in the range of about 0.1 μm to 10 μm, though other pore sizes are also contemplated. The overall dimensions of the membrane 40 include any size that is compatible with or otherwise based on the dimensions of segments 12a and 12b, such as about 1-100 mm by about 1-100 mm, though other overall dimensions are also contemplated. The thickness of the membrane 40 is generally in the range of about 10 μm to about 500 μm, and in some aspects, the thickness is about 20-50 μm. In some aspects, the thickness can be less than 1 μm or greater than 500 μm. It is contemplated that the membrane 40 can be made of a cured PDMS (poly-dimethylsiloxane), though other elastomeric materials like SEBS (styrene ethylene butylene styrene) and rigid materials like polycarbonate or polyester polymers are also contemplated. The membrane 40 separates the upper microchannel 34 from the lower microchannel 36 in an active region 37, which includes a bilayer of cells in the illustrated embodiment. In particular, a first cell layer 42 is adhered to a first side of the membrane 40, while a second cell layer 44 is adhered to a second side of the membrane 40. The first cell layer 42 may include the same type of cells as the second cell layer 44. Or, the first cell layer 42 may include a different type of cell than the second cell layer 44. And, while a single layer of cells is shown for the first cell layer 42 and the second cell layer 44, the first cell layer 42 and the second cell layer 44 may include multiple cell layers. Further, while the illustrated embodiment includes a bilayer of cells on the membrane 40, the membrane 40 may include only a single cell layer disposed on one of its sides.

The OOC device 10 is configured to simulate a biological function that typically includes cellular communication between the first cell layer 42 and the second cell layer 44, as would be experienced in-vivo within organs, tissues, cells, etc. Depending on the application, the membrane 40 is designed to have a porosity to permit the migration of cells, particulates, media, proteins, and/or chemicals between the upper microchannel 34 and the lower microchannel 36. The working fluids with the microchannels 34, 36 may be the same fluid or different fluids. As one example, as device 10 simulating a lung may have air as the fluid in one channel and a fluid simulating blood in the other channel. When developing the cell layers 42 and 44 on the membrane 40, the working fluids may be a tissue-culturing fluid.

In one aspect, the active region 37 defined by the upper and lower microchannels 34, 36 has a length of about 0.1-10 cm, a height of about 10-10,000 μm, and a width of about 10-2000 μm. The OOC device 10 preferably includes an optical window that permits viewing of the fluids, media, particulates, etc. as they move across the first cell layer 42 and the second cell layer 44. Various image-gathering techniques, such as spectroscopy and microscopy, can be used to quantify and evaluate the effects of the fluid flow in the microchannels 34, 36, as well as cellular behavior and cellular communication through the membrane 40. More details on the OOC device 10 can be found in, for example, U.S. Pat. No. 8,647,861, which is owned by the assignee of the present application and is incorporated by reference in its entirety. Consistent with the disclosure in U.S. Pat. No. 8,647,861, in one preferred aspect, the membrane 40 is capable of stretching and expanding in one or more planes to simulate the physiological effects of expansion and contraction forces that are commonly experienced by cells.

Micro- and mesofluidic devices and membranes compatible with the clamping can be fabricated from a variety of materials, including plastics, glass, silicones, biological materials (e.g., gelatin, collagen, chitosan, and others).

The OOC device 10 described in FIGS. 1 and 2 has multiple microchannels on either side of the membrane. However, the unique geometries of the seeding channel and the microchannel can be applied to microfluidic devices having only a single fluid path, whereby only part of the path includes a cellular attachment region (e.g., a membrane) that is preceded by a seeding region that feeds into the cellular attachment region.

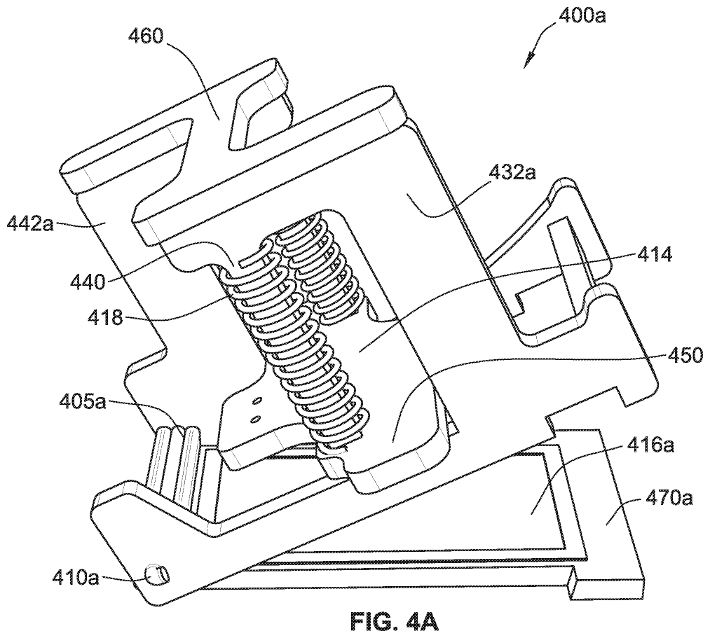

Turning now to FIGS. 3 through 9 various exemplary systems are illustrated that can be used for clamping microfluidic devices or devices that may be used in microfluidic systems.

A clamping system for a microfluidic device can include a base for engaging a first side of the microfluidic device. A plurality of elongated posts can extend upwardly from the base. A compression plate is movably coupled to the plurality of elongated posts such that the compression plate is vertically slidable along the posts. The compression plate engages a second side of the microfluidic device. A compression device provides compressive forces generally in a direction along the elongated posts. The compression device is operatively connected to the compression plate such that the compressive forces create a substantially uniform pressure on the second side of the microfluidic device.

In some aspects, the compression plate includes at least one inlet access hole that substantially aligns with a corresponding fluid inlet on the microfluidic device and at least one outlet access hole that substantially aligns with a corresponding fluid outlet on the microfluidic device. The inlet access hole and the outlet access hole each securely hold any fluid connectors disposed within the hole and are connected to a clamped microfluidic device.

A bottom surface area of the compression plate is greater than a top surface area of the microfluidic device. The base has a width such that the compression plate width is greater than the base width. The compression plate further includes two finger nubs or tabs protruding from a central portion of the compression plate and extending beyond the base such that a compression plate width with the finger nubs is greater than the base width.

In some aspects, the plurality of elongated posts is substantially parallel and the compression plate includes a plurality of apertures operative to allow an elongated post to pass through a respective aperture. The plurality of elongated posts supports the compression device. The compression device can include at least one spring extending around an outer boundary of at least one of the plurality of elongated posts.

It is contemplated that a maximum compressive force that is provided to the microfluidic device is determined based on a type of membrane present in the microfluidic device, a type of cell tissue present in the microfluidic device, and to minimize collapse of microfluidic channels. In some aspects, the compressive forces provided can range from approximately 5 kPa (0.7 psi) to approximately 200 kPa (29 psi).

In some aspects, a compression device, such as springs, apply approximately one kilogram of force in total to a compression plate. The approximate one kilogram force is substantially uniformly applied to a surface of a microfluidic device having a top surface area in the range of approximately 100 to 2000 mm², though in some aspects the range is more typically about 300 to 800 mm².

The compressive force to be applied is determined based on a type of membrane present in the microfluidic device, a type of cell tissue present in the microfluidic device, and to minimize collapse of microfluidic channels. It is desirable that the amount of force or pressure applied by a compression plate to a microfluidic device keep a microfluidic device sealed or properly sandwiched between the compression plate and a base while not being so extreme as to cause the collapse of the microfluidic channels or to prevent desired gas exchange. The pressure should allow cells to grow in the microfluidic channels while preventing escape.

Clamp system components can be made from different types of materials, including PMMA (e.g., acrylic), thermoplastics, thermoset polymers, other polymer materials, metals, wood, glass, or ceramics. In some aspects, the components of the clamping system can be fabricated using injection molding, casting, die cutting, laser machining and cutting, milling, or 3D printing. It is also contemplated that the bottom of the clamping system (such as, the base elements 370, 470a, 470b, 570, 670) can be fabricated to be of a certain thickness, or can be completely absent other than a perimeter frame, to allow for short working distance microscopic imaging for higher magnification and resolution.

One or more of the illustrated embodiments in FIGS. 3 through 9 include a compression device comprising two springs that provide a substantial uniform or equalized pressure to a compression plate (such as, elements 314, 414, 514, 614, 714, 714′, 814, 914, 924), which is a mobile part of the clamping system that moves easily up and down and in other axes to allow for easy of the clamping system. The compression plate can be modified in area, shape, thickness, or material. It is contemplated that the compression plate can include aspects where the compression plate includes generally smooth, even, and/or flat surfaces. It is also contemplated that the compression plate can include aspects where the compression plate includes generally uneven, indented, unlevel, and/or irregular surfaces. It is further contemplated that in some aspects the compression plate may be broad and level with little height or thickness. Yet, in other aspects, the compression plate may have a more significant height or thickness or a shape different from a traditional plate-like structure.

The use of springs in a clamping system can be desirable because springs constants can provide for a wide range of translation distances and forces and are versatile for situations where a clamping system may be positioned upside down for extended periods of time.

As discussed in more detail below, the compression plate covers all or a substantial portion of a top surface of a microfluidic device (e.g., a “chip”) and includes access holes for fluidic connections. A glass slide is integrated into the clamp system to provide a rigid support for the microfluidic device which improves pressure distribution for flexible devices (such as those made from PDMS silicone) while enabling good optical access for macroscopic, visual, or microscopic imaging that may be desirable through viewing portions of the clamp system.

It is contemplated that described clamping system facilitates the use or positioning the clamping system in an upside down position. This can be a particularly desirable feature during cell seeding of the underside of a membrane (e.g., such as membrane 40), commonly done during cell co-culture. Microfluidic devices when clamped in the described clamping systems are further stabilized which minimizes contamination of the fluidic connections by keeping the microfluidic device a distance above the incubator, bench, or other surfaces.

Exemplary vertical posts, such as the elongated or vertical posts 330, 340, 440, 540, 630, 640, can be loaded with varying amounts of force to obtain the desired pressure. While the design of the posts are illustrated with square or rectangular cross-section, the shape of the post itself can be that of any geometric entity that allows a compression device, such as a spring, to apply the needed force to the compression plate.

The exemplary compression plates, such as elements 314, 414, 514, 614, 714, 714′, 814, 914, 924 illustrated in FIGS. 3 through 9, are configured to apply a substantially uniform pressure across a membrane of a microfluidic device that is placed in the clamp system. Almost the whole force from the compression device (e.g., the springs, such as 317, 318, 418, 717, 718, 818) is applied over the entire portion of the compression plate. In the illustrated embodiments, of FIGS. 3 through 9, the compression device is desirably centered about the mid-point of where the microfluidic device is expected to be placed. In some aspects, the compression plate is configured to be loosely connected on the vertical or elongated posts, such that the compression plate can be displaced forward and backwards, and left and right, which further allows for a substantially uniform pressure to be applied to a microfluidic device, even if the microfluidic device is not flat. Furthermore, the exemplary spring-based compression device also allows for operational efficiencies by providing an easy way to apply sealing pressures to a microfluidic device, while providing a simple way to move the compression plate up or down as changes need to be made to the microfluidic device. While two springs are generally illustrated in the embodiments of FIGS. 3, 4A, and 7-9, it would be understood that more or fewer springs can be used to apply the force to the compression plate.

It is also contemplated that pressure on the compression plate can be adjusted using different springs (with various spring constants or lengths), spacers between the clamping system and the fluidic device, and fluidic device materials with different stiffnesses, any of which would produce different amounts of force in sealing the membrane of a microfluidic device. In some aspects, the spring force can be controlled by varying the maximum spring extension using an adjusting screw or other device for altering a spring extension distance.

In some aspects, a compression plate can include pressure-focusing features (e.g., areas with stiffer materials, areas with softer materials, areas with sloped features) to increase pressure in specific areas of the clamped fluidic devices where it may be desirable to have additional robustness, such as around holes or other mechanically-critical features.

A compression device for the clamping system can include alternatives to springs. For example, hydraulic or pneumatic compression systems are contemplated. It is also contemplated that for rigid microfluidic devices compliant gaskets can be used. For example, the clamping system embodiments of the present disclosure can be fitted with a compliant gasket that has a level of springiness to it rather than a spring itself. The compliant gasket materials would create an interface between the compression plate and the microfluidic device. It is also contemplated that in some aspects a compression device can utilize geometric shapes, such as cantilevered beams, as part of the device design to provide compressive force resulting from the case material flexure or compression. In some aspects, the compressive force can also be provided with magnetic or electromagnetic systems.

In some aspects, an exemplary spring-loaded clamping system is used to provide compression to a biocompatible polymer that uniformly seals an open region of a microfluidic device without adhesives. Such sealing can be further improved by including an elastomeric, pliable, or soft material in at least one of a cover (e.g., compression plate 614, 714, 714′, 814, 924; a separate layer between the compression plate and the microfluidic device that permits but also limits the exposure of an open region in a microfluidic device) or top surface (e.g., 613) of a microfluidic device (e.g., 612, 712, 812, 912). Different forms of gasketing and sealing known in the art are contemplated. An advantage certain aspects that employ a clamping system is that such systems facilitate the application, removal, and potentially the reapplication of a lid or cover, which may desirably allow access to an open region of the microfluidic device after it would normally be covered for experimentation purposes. Allowing limited access to an open region of the microfluidic device during experimentation can be useful, for example, in (i) the application of topical treatment, aerosol, additional cells or other biological reagents, (ii) change of fluidic (e.g. tissue-culture media), (iii) sampling of fluidic or solid matter, or (iv) imaging using optical or other techniques. The option to reposition the lid or cover, or apply a different cover, further permits the continued use of the device (e.g. in a biological experiment). Alternatively, the lid or cover may be removed at the end of the device's use to permit sampling that may be destructive, such as taking biopsies or otherwise removing samples, staining, fixing, or imaging.

In some aspects, an exemplary microfluidic device includes an open-top microfluidic device (e.g., a microfluidic device including a top surface with an open region and a removable cover) disposed within an exemplary clamping device, and includes open-top microfluidic devices such as those disclosed in U.S. Application No. 62/263,225, filed Dec. 4, 2015, entitled “Open-Top Microfluidic Devices and Methods for Simulating a Function of a Tissue”, and open-top microfluidic devices disclosed in a PCT application filed with the U.S. Receiving Office on Dec. 2, 2016, entitled “Open-Top Microfluidic Devices and Methods for Simulating a Function of a Tissue”, identifiable by Attorney Docket No. 002806-83890 and International Application No. PCT/US2016/064798, both patent application disclosures being hereby incorporate by reference herein in their entireties. A clamping device can be desirable because no glue or bonding is needed to hold the various layers of the microfluidic device together. The clamping device applied to an open-top microfluidic device optionally allows efficient removal of the removable cover during an experiment. The clamping device for the microfluidic device can include an optional base for engaging a first side (e.g., the bottom side) of the microfluidic device. In some aspects, a plurality of elongated posts can extend upwardly from the base. A compression plate, which may be flat or may in some aspects be uneven or in some aspects of a more substantial thickness or in some aspects shaped differently from what might be traditionally understood as a plate-like structure, is movably coupled to the plurality of elongated posts such that the compression plate is vertically slidable along the posts. In some embodiments, the compression plate engages a second side (e.g., the top side) of the microfluidic device; in other embodiments, the compression plate retains a cover to the microfluidic device. A compression device provides compressive forces generally in a direction along the elongated posts. The compression device (e.g., springs, elastomers, flextures, etc.) is operatively connected to the compression plate such that the compressive forces create a substantially uniform pressure on the second side (e.g., the top side) of the microfluidic device. Clamping device components can be made from different types of materials, including PMMA (e.g., acrylic), thermoplastics, thermoset polymers, other polymer materials, metals, wood, glass, or ceramics. In alternate embodiments, the compressive plate may be held in place using a retention mechanism including one or more of screws, clips, tacky/sticky materials, other retention mechanisms known in the art, or the combination of any of these mechanisms and/or the aforementioned compression device. In some embodiments, the retention mechanism retains the compressive plate with respect to or against the base. In alternate embodiments, the retention mechanism retains the compression plate with respect to or against the microfluidic device. For example, screws can be used to fasten the compression plate against the microfluidic device with the corresponding threaded holes included in the microfluidic device. As another example, the compression plate can include a clip feature (as a retention mechanism) that clips into a suitable receiving feature of the microfluidic device. In some embodiments, the compression plate comprises a cover for an open area included in the microfluidic device. In other embodiments, the compression plate retains an additional substrate that comprises a cover for an open area included in the microfluidic device.

In some aspects, a compression plate may include at least one access hole that substantially aligns with a corresponding fluid port (e.g., fluid inlet hole 681, 682 or fluid outlet hole 683, 684) on the microfluidic device or an optional cover. In some embodiments, the access hole securely holds or comprises a fluid connector. Such a fluidic connector may be beneficial in fluidically interfacing with the microfluidic device or optional cover without necessitating that the connector be included in the microfluidic device or optional cover.

A bottom surface area of the compression plate may be greater or smaller than a top surface area of the microfluidic device. In some aspects, the base can have a width such that the compression plate width is greater than the base width. The compression plate can further include finger nubs or tabs protruding from a central portion of the compression plate and extending beyond the base such that a compression plate width with the finger nubs is greater than the base width.

In aspects that include elongated posts, it is contemplated that the plurality of elongated posts are substantially parallel and the compression plate includes a plurality of apertures operative to allow an elongated post to pass through a respective aperture. The plurality of elongated posts supports the compression device (e.g., springs). The compression device can include at least one spring extending around an outer boundary of at least one of the plurality of elongated posts. In some aspects, a compression device comprises two springs that provide a substantial uniform or equalized pressure to a compression plate where the compression plate is a mobile part of the clamping device that moves easily up and down (or along other axes) to allow for easy manipulation of the clamped system. For example, the use of springs in a clamping device can be desirable because spring constants can provide for a wide range of translation distances and forces and are versatile for situations where a clamping device may be positioned upside down for extended periods of time. The compression plate can be modified in area, shape, thickness, or material. It is contemplated that the compression plate can include aspects where the compression plate includes generally smooth, even, and/or flat surfaces. It is also contemplated that the compression plate can include aspects where the compression plate includes generally uneven, indented, irregular, and/or unlevel surfaces. It is further contemplated that in some aspects the compression plate may be broad and level with little height or thickness. Yet, in other aspects, the compression plate may have a more significant height or thickness or a shape different from a traditional plate-like structure.

It is contemplated that a maximum compressive force that is provided to the microfluidic device by the clamping device is determined based on the force required to create a fluidic seal between the compression plate or optional cover and the microfluidic device (if such a seal is desired), and the propensity for the collapse of microfluidic channels or chambers within the microfluidic device or optional cover. In some aspects, the compressive forces provided can range from approximately 50 Pa (approximately 0.007 psi) to approximately 400 kPa (approximately 58 psi). In some aspects, the compressive forces provided can range from approximately 5 kPa (0.7 psi) to approximately 200 kPa (29 psi). In some embodiments, it is desirable that the amount of force or pressure applied by a compression plate to a microfluidic device keep a microfluidic device sealed or properly sandwiched between the compression plate and a base while not being so extreme as to cause the collapse of the microfluidic channels or to prevent desired gas exchange.

A glass slide or other transparent window (e.g. made of PMMA, polycarbonate, sapphire) can be integrated into the clamp device to provide a rigid support for the microfluidic device which improves pressure distribution for flexible devices (such as those made from PDMS silicone) while enabling good optical access for macroscopic, visual, or microscopic imaging that may be desirable through viewing portions of the clamp system.

It is contemplated that the described clamping device can facilitate the use or positioning of the device in an upside down position. This can be a particularly desirable feature during cell seeding of the underside of a chip membrane, commonly done during OOC co-culture.

A compression device for the clamping system can include alternatives to springs or other aforementioned compression devices or retention mechanisms. For example, hydraulic or pneumatic compression systems are contemplated. It is also contemplated that for rigid microfluidic devices compliant gaskets can be used. For example, the clamping device can be fitted with a compliant gasket that has a level of springiness to it rather than a spring itself. The compliant gasket materials would create an interface between the compression plate and the microfluidic device or between an optional cover and the microfluidic device. It is also contemplated that in some aspects a compression device can utilize geometric shapes, such as cantilevered beams, as part of the device design to provide compressive force resulting from the case material flexure or compression. In some aspects, the compressive force can also be provided with magnetic or electromagnetic systems.

Referring now to FIGS. 3A-3C, an exemplary clamping system is illustrated including an isometric view (FIG. 3A), a top view (FIG. 3B), and a side view (FIG. 3C) for the device. The clamping system 300 may be used for microfluidic devices or as part of a microfluidic system. The clamping system includes a base 370 that has a plurality of elongated side support structures, such as 332 and 342, protruding upwardly from a first edge 372 and an opposing second edge 374. At least two of the plurality of elongated side support structures each includes a vertical post, such as posts 330 and 340. A top cover 360 rigidly connects at least two of the plurality of elongated side structures. The plurality of elongated side structures connect at opposing sides 364, 366 of the top cover 360.

A compression plate 314 is movably coupled to at least two of the vertical posts 330, 340 such that the compression plate 314 is vertically slidable (see vertical arrows in FIG. 3A) between the base 370 and the top cover 360 along a long axis of each of the at least two vertical posts 330, 340. The compression plate 314 including at least one inlet access hole, such as 391, 392, that substantially aligns with a corresponding fluid inlet (not shown) on a microfluidic device (not shown) and at least one outlet access hole, such as 393, 394, that substantially aligns with a corresponding fluid outlet (not shown) on the microfluidic device. A compression device (such as springs 317, 318) provide compressive forces along each of the long axes of the at least two vertical posts 330, 340. The compression device is operatively connected to the compression plate 314 such that the compressive forces are applied directly to the compression plate 314. The compression plate is further operative to slide upwardly (see the vertical arrow pointing up in FIG. 3A) along the at least two vertical posts 330, 340 until the compression device is in a fully compressed state and operative to slide downwardly (see the vertical arrow pointing down in FIG. 3A) along the at least two vertical posts 330, 340 so that the compression plate 314 applies a substantially uniform pressure to a top surface (not shown) of a microfluidic device (not shown) positioned between the compression plate 314 and the base 370.

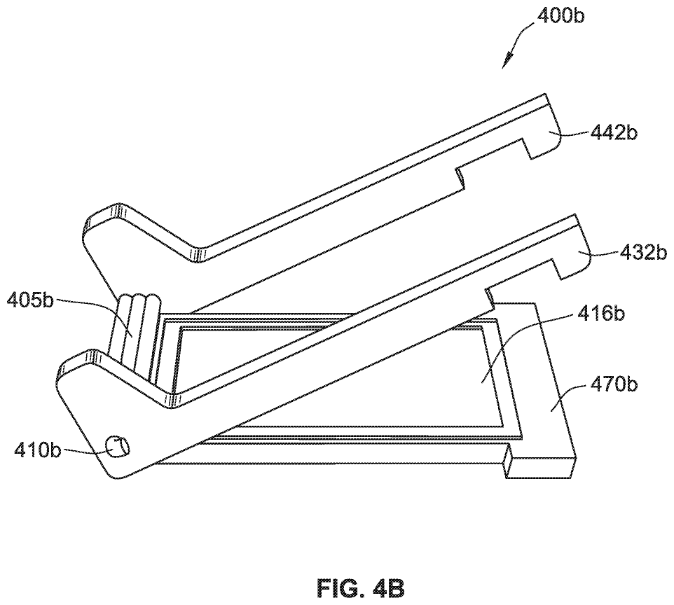

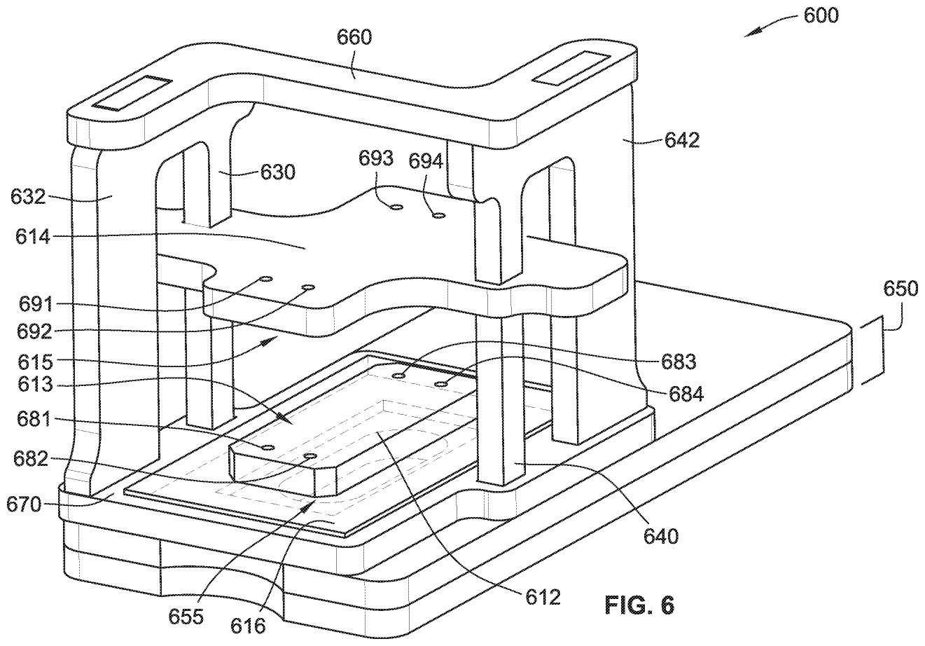

The base 370 of the clamping system 300 can include an aperture 355 extending therethrough. The aperture can allow for the placement of a glass slide (such as glass slide 416a, 416b, or 616 in FIGS. 4A, 4B, and 6) into the base 370. In some aspects, the glass slide substantially covers the aperture and is positioned to support the microfluidic device (not shown) between the glass slide and the compression plate 314.

The compression plate 314 can have a bottom surface 315 that has an area that is greater than the area of a top surface of the clamped microfluidic device. Furthermore, in some aspects, the base 370 and top cover 360 each have a width, W1. The compression plate 314 can also include two finger nubs 350, 352 laterally protruding from a central portion 362 of the compression plate 314 and extending beyond the base 370 and top cover 360 such that the compression plate width, W2, is greater than the width of both the base and the top cover.

In some aspects, the two vertical posts, such as posts 330 and 340, are substantially parallel. The compression plate 314 can include two apertures each operative to allow one of the vertical posts to pass therethrough. The vertical posts support the compression device, such as springs 317, 318. The springs 317, 318 can extend around an outer boundary of their respective vertical posts 330, 340 and are positioned around the vertical post between the compression plate 314 and the top cover 360.

The compression device that includes springs 317 and 318 in clamping system 300 are configured so that at least one spring is always in a compressed state such that compressive forces are constantly applied along each of the long axes of the at least two vertical posts 330, 340 and to the compression plate 314.

In some aspects, the claiming systems, such as systems 300, 400a, 500, and 600, have elongated side structures where each has a vertical post with only one of the vertical posts for each elongated side structure being connected to the compression plate. The vertical posts, such as posts 330, 340, are loosely connected to the compression plate 314 and extend upwardly from the two opposing edges 372, 374 of the base 370 at a central portion 362 of the clamp system, such as system 300. The vertical posts, such as elements 330, 340, that are connected to the compression plate can be parallel to each other and defining a plane orthogonal to the opposing side edges, such as elements 372, 374, and bisect the base, such as elements 370.

In some aspects, a compression plate, such as plate 314, can be locked in place using a snap-fit that occurs after placement of a microfluidic device into the clamp system. The snap-fit locks the compression plate in place to avoid motion or release of the clamped microfluidic device.

The compression plate 314 can include two finger nubs or tabs 350, 352 that protrude from opposing sides of a central portion 362 of the compression plate. The finger nubs or tabs extend beyond the width of the base 370 and the width of the top cover 360 such that a compression plate width, W2, including the fingers nubs 350, 352 is greater than the width of both the base and the top cover. The finger nubs 350, 352 are also laterally off-set from each other with the distance of the off-set being at least equal to the width, W3, of the individual fingers nubs. 350, 352.

In the exemplary aspect of the clamping system of FIG. 3 for a microfluidic device, such as the 00C devices described in FIGS. 1 and 2, the system can be sized such that the dimension W1 ranges from about 0.5 to about 10 cm; the dimension L ranges from about 0.5 to about 10 cm; the dimension H ranges from about 0.2 to about 10 cm; the dimension W2 range from about 0.5 to about 15 cm; the dimension W3 range from about 0.1 to about 2 cm; and the dimension D′ ranges from about 0.1 to about 2 cm. In some aspects, it is further contemplated that the range of the dimensions can be broader with W1 ranging from about 0.1 to about 50 cm; L ranging from about 0.2 to about 50 cm; H ranging from about 0.1 to about 10 cm; W2 ranging from about 0.1 to about 50 cm; W3 ranging from about 0.1 to about 10 cm; and D′ ranging from about 0.1 to about 10 cm. Factors that can determine the above described dimensions include how many chips are to be clamped in one unit of a clamp, the desired dimensions and spacing for a cell culture, the ease of handling of the clamping systems, the ability to make visual observations under a microscope, and other processes that may be desired to be carried out on the clamped chip(s).

In a desirable aspect for a top cover, including top covers 360, 460, 560, and 660, is should be configured as illustrated, for example, to allow for the clamping systems to be turned upside down, yet still provide a flat or level support for the clamped microfluidic device. This can be useful to allow for uniform cell feeding when cells are being grown or otherwise provided on the underside of a membrane, such as element 40.

Referring now to FIG. 4A, another exemplary clamping system is illustrated that includes a pivot about a base. The clamping system 400a may be used for microfluidic devices or as part of a microfluidic system. In some aspects, clamping can be accomplished using a hinged, clam-shell-like system, such as that in clamping system 400a. A clamshell embodiment can be desirable because the loading of a microfluidic device is easier and the use of greater compression force can be achieved without affecting usability.

The clamping system 400a includes a hinge pin 405a that connects a base 470a to elongated side support structures 432a, 442a that are rigidly connected using a top cover 460. The hinge pin 405a can extend out of a hole in the side support structure and may include a locking bend to keep the pin from sliding out, and thus, minimizing the chance the base 470a and rigidly connected side support structures unintentionally separate. The base 470a has a glass slide 416a positioned in the center of the base 470a above an aperture that defines a viewing window through the base 470a. A microfluidic device (not shown) can be placed on the glass slide 416a when the clamping device 400a is in an open position with the side support structures pivoting in a counter clockwise direction about the hinge pin 405a. After the microfluidic device is placed in the desired location on the glass slide 416a, the side support structures 432a, 442a are rotated in a clockwise direction so that a compression plate 414 swings onto and engages the top surface of the microfluidic device. This step may result in some compression of the compression device, such as spring 418, in applying a force to the compression plate 414. The rigidly connected side support structures 432a, 442a, can then be locked to the base 470a and the desire activity can be completed for the clamped microfluidic device. It is also contemplated that the compression plate can have finger tabs extending laterally from the compression plate to allow a clamping system user to manipulate the compression plate in an upward and downward direction as guided by vertical posts, such as post 440, which extends through one or more apertures in the compressions plate 414.

Referring now to FIG. 4B, another exemplary clamping system is illustrated that includes a pivot along the base, similar to the system illustrated in FIG. 4A. The clamping system 400b may be used for microfluidic devices or as part of a microfluidic system. In some aspects, clamping is accomplished using a hinged, clam-shell-like system, such as that in clamping system 400b. Similar to FIG. 4A, a clamshell embodiment can be desirable because the loading of a microfluidic device is easier and the use of greater compression force can be achieved without affecting usability.

The clamping system 400b includes a hinge pin 405b that connects a base 470b to elongated side support structures 432b, 442b. The hinge pin 405b can extend out of a hole in the side support structure and may include a locking mechanism, such as a locking bend to keep the pin from sliding out, and thus, minimizing the chance the base 470b and connected side support structures unintentionally separate. The base 470b has a glass slide 416b positioned in toward the center of the base 470b above an aperture that defines a viewing window through the base 470b. A microfluidic device (not shown) can be placed on the glass slide 416b when the clamping device 400b is in an open position with the side support structures pivoting in a counter clockwise direction about the hinge pin 405b. After the microfluidic device is placed in the desired location on the glass slide 416b, the side support structures 432b, 442b are rotated in a downward direction so that a compression plate (not shown), secured directly or indirectly to the side support structures 432b, 442b, swings onto and engages the top surface of the microfluidic device. The side support structures 432b, 442b, can then be locked to the base 470b and the desire activity can be completed for the clamped microfluidic device. It is also contemplated that the compression plate (not shown) secured to the side support structures can be configured to allow a clamping system user to manipulate the compression plate in an upward and downward direction through the use of guides adjacent to or that are a part of the compression plate.

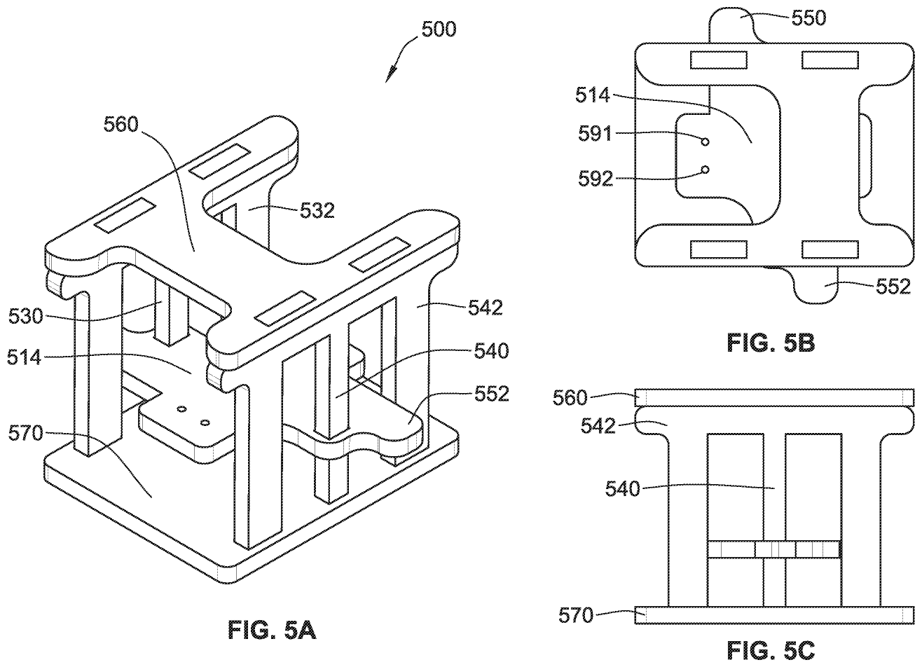

Referring now to FIGS. 5A-5C, another exemplary clamping system is illustrated including an isometric view (FIG. 5A), a top view (FIG. 5B), and a side view (FIG. 5C) for the device. The clamping system 500 may be used for microfluidic devices or as part of a microfluidic system.

The clamping system 500 includes a base 570 with elongate side support structure 532, 542 oriented along a first edge and an opposing second edge of the base 570. The side support structure 532, 542 can extend upwardly from the base 570. The side support structures 532, 542 are further connected to a top support 560 that rigidly connects the two structures 532, 542. The side support structure can further include vertical posts 530, 540 that extend through two respective apertures in a compression plate 514 that moves up and down, as guided along posts 530, 540. A compression device (not shown) applies a force to the compression plate as similarly discussed for other clamping system embodiments in the present disclosure. It is also contemplated that the compression plate 514 can have finger tabs 550, 552 that extend laterally from the compression plate 514 and allow a clamping system user to manipulate the compression plate 514 in an upward and downward direction as guided by vertical posts 530, 540 that extend through one or more apertures in the compression plate 514. The compression plate 514 can also include one or more access holes, such as elements 591 and 592, to allow for fluid connection to be made to a microfluidic device.

The clamping system illustrated in FIG. 3, when viewed from the top, has a top cover 360 appears to be shaped like a two-side “L” or an “S”. In the clamping system 500 illustrated in FIG. 5, the top structure 560 is shaped like an “H”. In some aspects, the “H” shaped top structure may be desirable where greater stability of the clamping system is preferred when the clamping system if turned upside down. In the clamping system 300 in FIG. 3, eliminating one or more of the side support structure components and transitioning the more “S” shaped top cover and more asymmetric shape can also be desirable to provide a user access from the sides of the clamping system to the fluidic connections. This can be beneficial when the chip is clamped and the fluidic connections need to be inserted with tubing or pins.

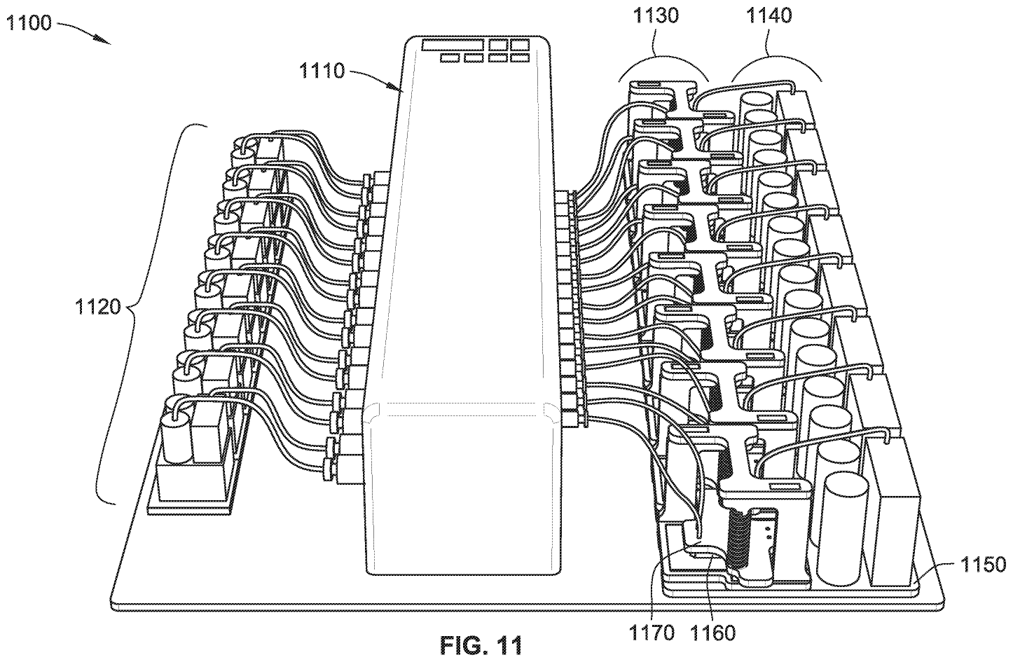

Referring now to FIG. 6, an exemplary microfluidic system is illustrated. The microfluidic system 600 includes an exemplary microfluidic device 612 that has been placed in a clamping device. The microfluidic system 600 is positioned on a farm cartridge 650, which is discussed in more detail in FIG. 11 for a cell culture incubator. The microfluidic device has a top surface 613 and an opposing bottom surface (not shown) that rests on a glass slide 616. In addition, the microfluidic device can include one or more fluid inlets, such as elements 681, 682 and fluid outlets, such as elements 683, 684, that are fluidly connected to one or more microchannels.

A clamp device portion of the microfluidic system 600 can include a base 670 that supports the glass slide 616. The base 670 can have an aperture 655 extending therethrough that provides a viewing window to observed activity in the microchannels of the microfluidic device. The viewing window is viewable from below the base when the clamping device is removed from the farm cartridge 650 and it permits imaging of a region of at least one microchannel. The clamping device further includes a moveable compression plate 614 having a bottom surface 615 for engaging the top surface 613 of the microfluidic device 612. The movable compression plate 614 is guided by vertical posts 630, 640 extending through two apertures in a compression plate 614 that are part of elongated side support structures 632, 642 oriented along a first edge and an opposing second edge of the base 670. The side support structures 632, 642 are connected by a top cover 660. The clamp device further includes a compression device (not shown) for urging the moveable compression plate 614 downwardly against the top surface 613 of the microfluidic device 612 to place a substantially uniform pressure on the top surface 613.

As the movable compression plate 614 is urged against the top surface of the microfluidic device, exemplary inlet access holes 691, 692 in the compression plate 614 align with exemplary fluid inlets 691, 682 of the microfluidic device 612. Similarly, exemplary outlet access holes 693, 694 in the compression plate 614 align with exemplary fluid outlets 683, 684 of the microfluidic device 612.

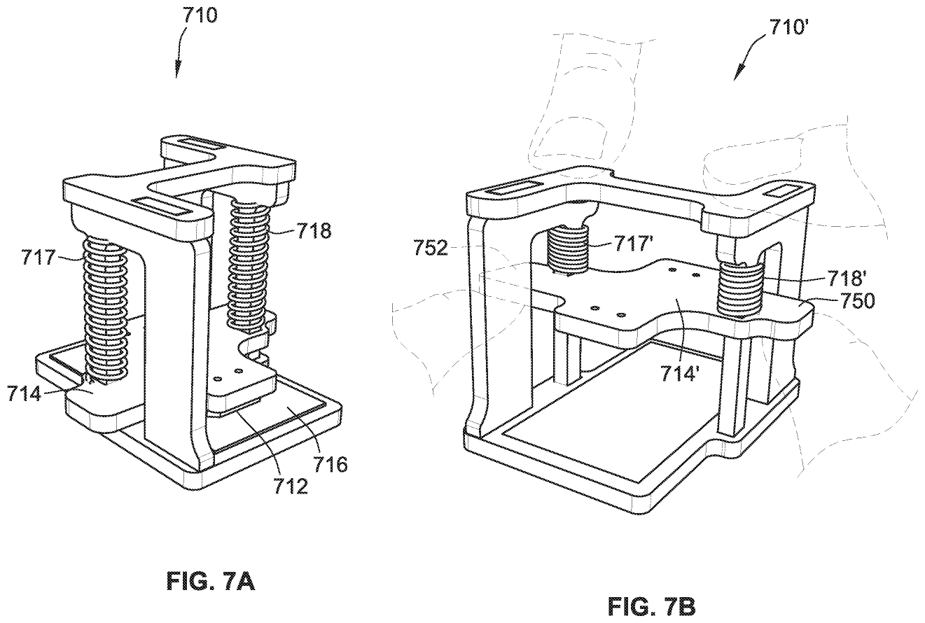

Referring now to FIGS. 7A and 7B, an exemplary microfluidic system is illustrated demonstrating a clamping device in a closed state (FIG. 7A) and in an open state (FIG. 7B) during the operation of the device.

A microfluidic system with a clamping device in a closed state 710 is shown in FIG. 7A with a compression plate 714 fully urged in a downward direction by compression devices, such as springs 717, 718, to clamp microfluidic device 712. The microfluidic device is sandwiched between the compression plate and a glass slide positioned on a base of the clamping device.

As the user of the microfluidic system desires to manipulate the microfluidic device, the compression plate 714′ is urged in an upward direct as illustrated by clamping device 710′. The user raises the compression plate 714′ using the finger tabs 750, 752 to fully compress the compression device, which includes springs 717′, 718′ in a fully compressed position. The microfluidic device can then be removed or otherwise manipulated once the compression plate 714′ is lifted.

In some aspects, a microfluidic system includes a microfluidic device 712 that includes a top part, a bottom part, a membrane between the top and bottom parts, and at least one microchannel at least partially defined by the membrane, such as elements 12a, 12b, 40, and 34 and 36 in FIG. 1. A clamp system of the microfluidic system includes a base for engaging the bottom surface of the microfluidic device. A movable compression plate 714, 714′ engages the top part of the microfluidic device 712 in a closed state and being released from the top surface in an opened state. A compression device, such as springs 717′, 718′ allow for controllably moving the moveable compression plate 714, 714′ between the closed state during operation of the microfluidic device 712 and the opened state allowing the top part, such as element 12a from FIG. 1, to be removed from at least one of the membrane and the bottom part, such as elements 40 and 12b from FIG. 1.

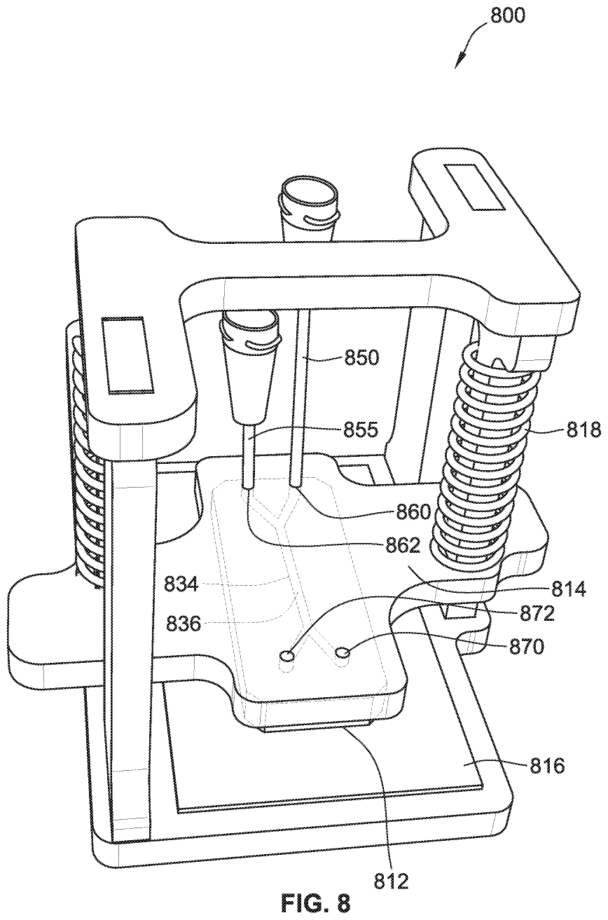

Referring now to FIG. 8, an exemplary microfluidic system is illustrated that includes a clamping device, a microfluidic device, and fluid connectors connected to the microfluidic device according to one embodiment.

The microfluidic system 800 includes a microfluidic device 812 a microfluidic device that includes a top surface in contact with a compression plate 814, a bottom surface in contact with a glass slide 816, one or more microchannels 834, 836, one or more fluid inlets, and one or more fluid outlets. A clamp system of the microfluidic system includes a base having a glass slide 816 for engaging the bottom surface of the microfluidic device 812. The movable compression plate 814 engages the top surface of the microfluidic device 812. A compression device, including one or more exemplary springs, such as element 818, urge the moveable compression plate 814 downwardly against the top surface of the microfluidic device 812 to place a substantially uniform pressure on the top surface. The compression plate 814 including one or more inlet access holes 860, 862 that substantially align with the fluid inlet on the microfluidic device 812 and one or more outlet access holes 870, 872 that substantially align with the fluid outlet on the microfluidic device 812. The inlet access hole(s) and the outlet access hole(s) each securely hold any fluid connectors, such as syringe needle connectors 850, 852 (e.g., long and short Leur connectors), disposed within the hole and connected to a clamped microfluidic device 812.

It is contemplated that fluidic connection can be made using various materials, such as metal tubing, plastic tubing, Luer and other connectors, or glass tubing.

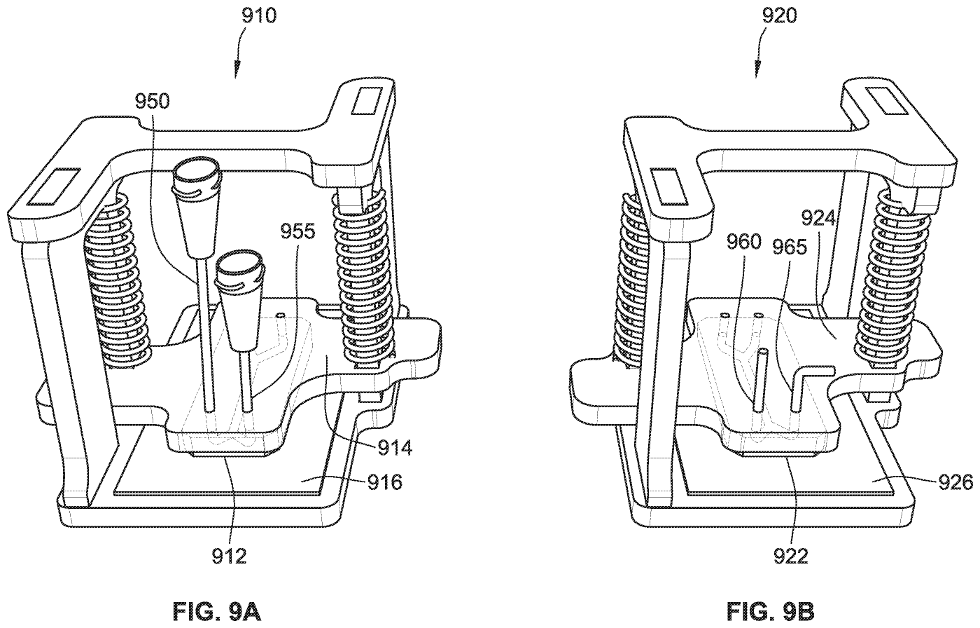

Referring now to FIGS. 9A and 9B, an exemplary microfluidic system is illustrated that includes a clamping device, a microfluidic device, and fluid connectors and needles connected to the microfluidic device according to one embodiment.

Each of the microfluidic systems 910, 920 include a microfluidic device 912, 922 that is clamped and fixed from below to a glass slide 916, 926 and from above by a compression plate 914, 924. Each of the microfluidic devices 912, 922 have one or more inlet access holes and one or more outlet access holes. In system 910, first fluid connectors 950, 955 are securely held their respective access holes. Similarly, second fluid connectors 960, 965 are also securely held by their respective access hole. First fluid connectors 950, 955 represent a long Leur connector and a short Leur connector syringe needles. Second fluid connectors 960, 965 represent a non-Leur straight needle and a non-Leur bent needle.

As illustrated in FIGS. 8 and 9 and in more detail in FIG. 6, the fluid connectors are secured into the access holes in the compression plate and the access holes are then aligned with the fluid inlets and outlets on the microfluidic device. In some aspects, alignment between these elements can be controlled by the insertion of rigid rods or tubing into two or more of the existing microfluidic device ports and aligning them with the corresponding compression plate (e.g., 814) holes. Alternatively, the 00C chip can be fabricated such that certain marks or impressions on the outer surfaces of or holes penetrating through segments 12a and/or 12b are fitted into pre-defined matching parts (e.g. lower surface of 814 or upper surface of 816) in the clamp. This allows the chip to sit in the clamp and holes and the inlets and outlets all stay aligned without the guide of connectors.

In the microfluidic devices clamped using the system described in the present disclosure, the holes the access holes in the compression plate are tight and can support the weight of the connector. The holes can therefore stabilize and securely hold the needle connectors along with allowing for interchangeability, even when the cells are in culture. Thus, the embodiment illustrated in FIG. 8 and also in FIG. 9, a user can change or modify the clamped fluidic system during cell culture and experimentation. For example, fluidic connectors can be interchanged, such as switching from Luer connectors during cell seeding to bent needles once the cells are confluent for perfusion of circulating cells. The clamping system allows connector exchange without increasing the chance of air bubbles during the exchange process. The travel distance of the compression plate allows chips to be assembled using parts of various thicknesses, so an experiment can readily use chips with different heights. Finally, a membrane clamped between two fluidic channels can also be easily removed for imaging and downstream assays and procedures, including implantation. As a result, the clamping system described by the present disclosure allow integration of microfluidic features not found in most current cell culture systems (e.g., fluidic shear, mechanical strain and compression, and rapid introduction of reagents) and readily transfer the treated tissue or cells into macro-scale conditions, and vice versa. Furthermore, while needles are described in the context of FIGS. 8 and 9, in some aspects, threaded access holes can be provided in the compression plate for receiving fluid connectors.

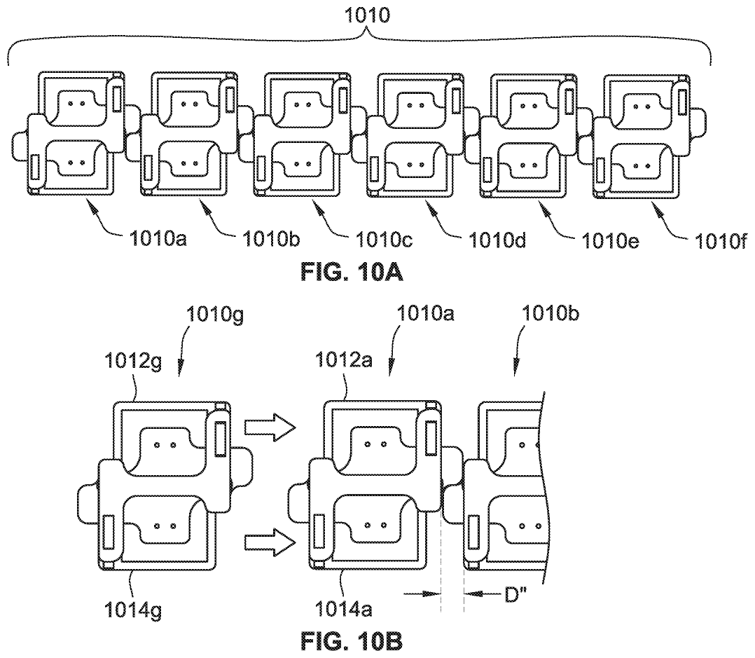

Referring now to FIGS. 10A and 10B, an exemplary clamp system farm is illustrated that may be used for microfluidic devices or as part or as part of a microfluidic system. The clamp system farm 1010 can use any one of the exemplary clamping systems contemplated by the present disclosure. A plurality of clamping systems are illustrated in FIG. 10A, such as clamping systems 1010a, 1010b, 1010c, 1010d, 1010e, 1010f, comprise the clamp system farm 1010. Each of the clamp system have the same configuration and are stacked side-by side such that the off-set of the finger nubs on one side of one clamp system and the finger nubs from another side of another clamp system allows the plurality of clamp systems to be stacked immediately next to each other, as illustrated in FIG. 10A. The distance between any two clamp systems is approximately the distance that one of the finger nubs protrudes from a compression plate of any one of the plurality of clamp systems.

FIG. 10B further illustrates the adding of a clamping system to the farm. Clamping system 1010g is added so that the front and back edges 1012g, 1014g of clamping system 1010g are in line with the front and back edges 1012a, 1014a of clamping system 1010a and the front and back edges 1012b, 1014b of clamping system 1010b. Each of the clamping systems are separated by a distance of approximately D″ (which can range from about 0.5 to about 5 cm) which is approximately the distance the finger nubs that have been described earlier (such as compression plates 314, 414, 514, 614, 714, 814, 914, 924, 1170) extend out from each side of the compression plates for each clamping system. As illustrated, the compression plates are not symmetric about their centerline(s) which allows for the offset nature of the finger nubs (e.g., wings, tabs) extending out from the compression plate as the clamping systems are stacked side-by-side with their front edges (such as 1012g and 1012a) and back edges (such as 1014g and 1014a) in line with each other. The configuration illustrated in FIGS. 10A and 10B saves space in the cell culture incubator, while still allowing enough of a finger nub or tab for lifting a clamping system's respective compression plate when you are decompressing the clamped microfluidic device. The offset of the finger nubs or tabs ranges from about 0.5 to about 10 cm.

Referring now to FIG. 11, an exemplary clamp system farm is illustrated that is part of a cell culture incubator. The clamp system farm 1010 for the cell culture incubator 1100 includes stacking a plurality of clamping systems side-by-side next to each other on a support 1150 that holds both the clamping systems and a plurality of cartridges 1140. The stacked clamping systems 1130 includes respective compression plates 1170 and microfluidic devices 1160, or “chips” that are clamped or loaded into and held by the clamp system. The plurality of cartridges 1140 includes hold cell medium reservoirs placed adjacent to a respective clamp system. A pump 1010, such as a peristaltic pump, can be used to generate negative or positive pressure ad flows through the microfluidic device through tubes and connectors connected to the microfluidic devices through access holes in the compression plates of the clamp systems. An effluent collector system 1120 allows for real-time collection of the flow-through from the microfluidic channels to allow for analysis of the fluid or so the fluids can be discarded.

The exemplary side-by-side stacked configured illustrated in FIG. 11 can be desirable because the stacking allowed the use of more microfluidic devices along a given width of a cell culture incubator, while providing for easy handling and use for each individual cartridge.

It is contemplated that in some aspects that a clamp system can have integrated fluid reservoirs, reagent reservoirs, pumps, or other actuators or valves. For example, in some aspects, the side support structures of a clamp system or the top cover can include supports that allow the connection of a reservoir or pump to the clamping system. The size of the reservoir(s) or pump can be smaller than those illustrated in FIG. 11 to allow for such connection to a clamp system.

Each of the above described aspects and obvious variations thereof are contemplated as falling within the spirit and scope of the claimed invention, which is set forth in the following claims. Moreover, the present concepts expressly include any and all combinations and subcombinations of the preceding elements and aspects.

1. A clamping system for a microfluidic device, the clamping system comprising:
a compression plate engaging a side of a microfluidic device; and
a compression device for providing compressive forces, the compression device being operatively connected to the compression plate such that the compressive forces create a pressure on the side of the microfluidic device,
wherein the compression plate includes at least one inlet access hole that substantially aligns with a corresponding fluid inlet on the microfluidic device and at least one outlet access hole that substantially aligns with a corresponding fluid outlet on the microfluidic device, the inlet access hole and the outlet access hole each securely holding any fluid connectors disposed within the hole and connected to a clamped microfluidic device.

2. (canceled)

3. The clamping system of claim 1, wherein a bottom surface area of the compression plate is greater than a top surface area of the microfluidic device.

4. The clamping system of claim 1, further comprising a base and elongated posts extending upwardly from the base, wherein the plurality of elongated posts are substantially parallel, the compression plate including plurality of apertures operative to allow an elongated post to pass through a respective aperture, the plurality of elongated posts supporting the compression device.

5. The clamping system of claim 4, wherein the compression device includes at least one spring extending around an outer boundary of at least one of the plurality of elongated posts.

6. The clamping system of claim 1, wherein a maximum compressive force that is provided is determined based on a type of membrane present in the microfluidic device, a type of cell tissue present in the microfluidic device, and to minimize collapse of microfluidic channels.

7. (canceled)

8. The clamping system of claim 1, further comprising:
a base for engaging a second side of the microfluidic device; and
a plurality of elongated posts extending upwardly from the base, wherein the compression plate is movably coupled to the plurality of elongated posts such that the compression plate is vertically slidable along the posts, the compressive forces being provided generally in a direction along the elongated posts

9. (canceled)

10. The clamping system of claim 1, wherein the compression plate has a shape that is generally broad or flat.

11. The clamping system of claim 1, wherein the compression plate includes uneven or unlevel surfaces.

12-29. (canceled)

30. A microfluidic system, comprising:
a microfluidic device including a top surface, a bottom surface, at least one microchannel, a fluid inlet, and a fluid outlet; and
a clamp system comprising (i) a moveable compression plate for engaging the top surface of the microfluidic device, and (ii) a compression device for urging the moveable compression plate downwardly against the top surface of the microfluidic device to place a substantially uniform pressure on the top surface, the compression plate including an inlet access hole that substantially aligns with the fluid inlet on the microfluidic device and an outlet access hole that substantially aligns with the fluid outlet on the microfluidic device.

31. The microfluidic system of claim 30, wherein the inlet access hole and the outlet access hole each securely hold any fluid connectors disposed within the hole and connected to the microfluidic device.

32. The microfluidic system of claim 30, further comprising a base for engaging the bottom surface of the microfluidic device.

33. The microfluidic system of claim 30, wherein the movable compression plate has a shape that is generally broad or flat.

34. (canceled)

35. The microfluidic system of claim 32, wherein the base includes a viewing window that permits imaging of a region of the at least one microchannel.

36-39. (canceled)

40. A method of clamping a microfluidic device, comprising:
a) providing (i) a microfluidic device comprising a side with ports in fluidic communication with at least one internal channel, and (ii) a clamping system for clamping said microfluidic device, the clamping system comprising a compression plate engaging a side of the microfluidic device and a compression device for providing compressive forces, the compression device being operatively connected to the compression plate; and
b) applying compressive forces with said compression device such that pressure is created on the side of the microfluidic device.

41. The method of claim 40, further comprising:
c) flowing culture media through the at least one internal channel.

42. (canceled)

43. The method of claim 40, wherein the compressive forces seal an open region of the microfluidic device.

44. The method of claim 43, wherein the compressive forces uniformly seal the open region of the microfluidic device, the uniform seal being formed without adhesives.

45. The method of claim 43, wherein said seal further comprises at least one cover.

46. (canceled)

47. The method of claim 43, further comprising a separate layer between the compression plate and the microfluidic device that permits a limited exposure of the open region.

48. (canceled)

49. The method of claim 47, wherein the limited exposure of the open region allows for activities selected from the group consisting of (i) the application of topical treatment, aerosol, additional cells or other biological reagents, (ii) change of fluidic media, (iii) sampling of fluidic or solid matter, (iv) imaging using optical or other techniques, (v) biopsies, (vi) removing samples, (vii) staining tissues or cells, viii) fixing tissues or cells, and (ix) imaging tissues or cells.

50-55. (canceled)
